(19) United States
(12) Patent Application Publication
(10) Pub. No.: US 2012/0093928 A1
(43) Pub. Date: Apr. 19, 2012

(54) ORAL METAXALONE COMPOSITIONS

(75) Inventors: Rajesh S. Shear, Subash Nagar (IN); Venkateshwaran Rathinasabapathy, Madurai (IN); Sumit Madan, New Delhi (IN); Vinod Kumar Arora, New Delhi (IN)

(73) Assignee: RANBAXY LABORATORIES LIMITED, New Delhi, Delhi (IN)

(21) Appl. No.: 12/672,631

(22) PCT Filed: Aug. 6, 2008
(86) PCT No.: PCT/IB2008/053168

§ 371 Date: Feb. 8, 2010

(30) Foreign Application Priority Data
Aug. 9, 2007 (IN) 1699/DEL/2007

(51) Int. Cl. A61K 9/00; A61P 21/02; A61P 29/00; A61K 31/421

(52) U.S. Cl. 424/484; 514/376

(57) ABSTRACT

The present invention relates to a pharmaceutical composition comprising low-dose metaxalone and one or more pharmaceutically acceptable polymer, as well as methods of preparing them.

### FIELD OF THE INVENTION

The present invention relates to a metaxalone composition for oral administration as well as methods of preparing them.

### BACKGROUND OF THE INVENTION

Metaxalone, 5-[(3,5-dimethyphenoxy) methyl]-2-oxazolidinone, is a skeletal muscle relaxant used to relieve the pain of muscle injuries, spasms, sprains, and strains. The mechanism of action of metaxalone in humans has not been established, but may be due to general central nervous system depression. It has no direct action on the contractile mechanism of striated muscle, the motor end plate, or the nerve fiber. The drug does not directly relax tense skeletal muscles in man. Metaxalone is indicated as an adjunct to rest, physical therapy, and other measures for the relief of discomforts associated with acute, painful musculoskeletal conditions.

U.S. Pat. No. 4,722,938, describes methods of using musculoskeletal relaxants such as metaxalone.

Metaxalone is marketed under the brand name SKELAXIN® (King Pharmaceuticals, Inc.) in 800 mg tablets. The general dosage for adults and children over 12 years of age is one 800 mg tablet, three to four times a day, amounting to about 2400-3200 mg per day (hereinafter referred to as ‘conventional daily dose’). According to American Hospital Formulary Service (AHFS) Drug Information, following oral administration of a single 800 mg dosage of metaxalone, mean peak plasma concentrations are attained in 2 hours. The onset of action is within 1 hour and duration of action is about 4 to 6 hours. It has a plasma half-life of 2-3 hours, thus requiring multiple dosing. Repeated administration of a very high dosage drug effectuates patient inconvenience and is very bothersome for ambulatory patients, leading to poor patient compliance.

Due to poor aqueous solubility metaxalone has poor bioavailability. Moreover it also shows food effect. U.S. Pat. Nos. 6,407,128 and 6,683,102, assigned to Elan Pharmaceuticals, disclose method of increasing the oral bioavailability of metaxalone by administration of an oral dosage form with food.

WO 2004/019937, filed by Sun Pharmaceutical, describes pharmaceutical composition comprising metaxalone and pharmaceutically acceptable excipients, characterized in that the pharmaceutical composition has enhanced oral bioavailability. The metaxalone that is used is a pharmaceutically acceptable solubility improved form, i.e. micronized metaxalone, salt form of metaxalone, high-energy crystalline form of metaxalone or amorphous metaxalone.

U.S. Patent Application No. 2005/0063913 A1 describes the composition comprising metaxalone particles having an effective average particle size of less than about 2000 nm and at least one surface stabilizer that is preferably adsorbed to or associated with the surface of the drug particles.

Since metaxalone is indicated for acute painful musculoskeletal conditions that require quick relief, it would be desirable to have a dosage form that maintains a balance between the amount of the drug released immediately and amount of drug released over an extended period. Further it is also desirable to have a dosage form that maintains the plasma level of metaxalone fairly constant, without any troughs and peaks.

The 3-4 times daily dosage that is standard for metaxalone therapy is highly inconvenient and adversely affects patient compliance. A formulation which reduces the dosing to at least twice daily frequency is highly desirable. However, a twice daily dose with normal delivery profile would have 1200-1600 mg of metaxalone per tablet, if provided in a single dosage form. Such a tablet would be bulky and difficult to ingest and would almost certainly lead to patient non-compliance.

In addition to the increased bulkiness, metaxalone itself is known to have side effects like dizziness, drowsiness or restlessness, lightheadedness, nausea or vomiting, stomach cramps or pain, headache, fever hives, anemia, hemolytic, itching, jaundice, agitation and rash. Life-threatening disease like anaphylaxis, extreme weakness, temporary vision loss and transient paralysis are very rare, but cannot be ruled out. It is also mentioned in the literature that these side-effects are amplified in the elderly. For these reasons, metaxalone is meant to be used in acute situations for short-term use only. Thus, decreasing the number of doses per day by increasing the strength is not desirable from both patient safety and compliance point of view. Rather, it would be advantageous to utilize lower dose of metaxalone while maintaining efficacy would be desirable for minimizing possible side effects and improving patient compliance.

We have developed a pharmaceutical composition comprising metaxalone that reduces the dose of metaxalone used per day. The dose in the composition may be reduced at least 10% of the conventional daily dose. That is, a total daily dose in the range of 2000 to 2900 mg (referred to herein as low-dose metaxalone) as against the conventional daily dose of 2400 to 3200 mg.

### SUMMARY OF THE INVENTION

In one general aspect there is provided a pharmaceutical composition for oral administration of a low-dose of metaxalone comprising metaxalone and one or more pharmaceutically acceptable polymers, wherein the dose of metaxalone is reduced by at least 10% of the conventional daily dose. Preferably the dose of metaxalone is reduced by at least 15% of the conventional daily dose.

Embodiments of the pharmaceutical composition may include one or more of the following features. For example, the composition comprises low-dose of metaxalone and one or more pharmaceutically acceptable polymers, wherein the dose of metaxalone is reduced by at least 10% of the conventional daily dose and when low-dose metaxalone pharmaceutical composition is administered twice a day, exhibits pharmacokinetic parameters comparable to the conventional dosage form (SKELAXIN® 800 mg) administered four times a day.

In one embodiment the polymer utilized is a rate controlling polymers and particularly the rate controlling polymers include one or more of hydrophilic polymers, hydrophobic polymers, or combinations thereof.

In another embodiment the composition of the present invention are in the form of matrix-type dosage form, a reservoir type dosage form, multiple unit dosage form or combinations of one or more dosage forms.

In another general aspect there is provided a pharmaceutical composition comprising metaxalone and one or more pharmaceutically acceptable polymers that can be conveniently administered twice in a day and will reduce the side effects like CNS effects and GI upset considerably, while retaining its efficacy.

It is yet another aspect to provide a pharmaceutical composition comprising metaxalone and one or more pharmaceutically acceptable polymer in a smaller sized dosage form that will ease the administration of dosage form in patients having difficulty in swallowing, especially the geriatrics.

The pharmaceutical composition may also include one or more pharmaceutically acceptable excipients acting in the capacity of fillers, binders, lubricants, glidants, colorants and flavoring agents.

In another general aspect to provide a pharmaceutical composition comprising metaxalone and one or more pharmaceutically acceptable polymer that have a desirable pharmacokinetic profile when administered to human subjects. The composition of the present invention has a more preferred pharmacokinetic profile as compared to conventional currently marketed metaxalone tablets, e.g. SKELAXIN® 800 mg. For example, the improved pharmacokinetic profile of the composition of the invention may produce the same pharmacokinetic profile as a conventional metaxalone tablets (i.e. SKELAXIN® 800 mg), but at a lower dose. Such an improved pharmacokinetic profile may also correspond to a metaxalone composition which requires less frequent dosing as compared to a conventional metaxalone tablets, such as once a day or twice day dosing. An improved or more preferred pharmacokinctic profile to the invention may also exhibit improved Tₘₐₓ, Cₘₐₓ and/or AUC profiles.

It is yet another aspect to provide a method of treating muscle spasm associated with painful musculoskeletal conditions in humans, by administering a pharmaceutical composition comprising metaxalone and one or more pharmaceutically acceptable polymer that provides a constant release profile for a period of time of up to at least twelve hours.

The method may further include administering other non-steroidal anti-inflammatory agents (NSAIDs), analgesics or other pharmaceutical agents.

The details of one or more embodiments of the invention are set forth in the description below. Other features, objects, and advantages of the invention will be apparent from the description and claims.

### DETAILED DESCRIPTION OF THE INVENTION

The inventors have developed a pharmaceutical composition for oral administration of a low-dose of metaxalone comprising metaxalone and one or more pharmaceutically acceptable polymers, wherein the dose of metaxalone is reduced by at least 10% of the conventional daily dose. Preferably the dose of metaxalone is reduced by at least 15% of the conventional daily dose.

In a standard dosage regimen an 800 mg immediate release dosage form of an active ingredient with a short half-life, such as metaxalone, may have to be administered to a patient three to four times a day to maintain adequate bioavailability of the drug to achieve therapeutic effect. This results in a series of serum concentration profiles in the patient in which there is a rapid increase of drug followed by a similar rapid decrease. Such rapid increases and decreases provide a patient with a short window of appropriate blood concentration of the medicament for optimum therapy.

A sustained release dosage form, on the other hand, may only have to be administered to a patient once every 12 hours to achieve therapeutic effect. Sustained release dosage forms generally control the rate of active drug absorption, so as to avoid excessive drug absorption while maintaining effective blood concentration of the drug to provide a patient with a consistent therapeutic effect over an extended duration of time, i.e., these dosage forms provide a uniform concentration of drug at the absorption site for extended period of time and thus after absorption allow maintenance of plasma concentrations within a therapeutic range, which minimizes the side effects and reduces the frequency of administration.

The pharmaceutical composition comprising metaxalone of the present invention, when ingested orally, may induce statistically significantly lower mean fluctuation index in the plasma than an immediate release composition of metaxalone while maintaining bioavailability substantially equivalent to that of the immediate release composition of metaxalone. Upon oral ingestion, maximum peak concentrations of the sustained release metaxalone compositions may be statistically significantly lower than those produced by an immediate release pharmaceutical composition, and an area under the concentration-time curve and the minimum plasma concentration may be substantially equivalent to that of the immediate release pharmaceutical composition.

The pharmaceutical composition of the present invention comprises low-dose of metaxalone and one or more pharmaceutically acceptable polymer, which can be advantageously administered twice daily. The composition has reduced side effects while retaining its complete efficacy.

The polymer used in the composition is rate controlling polymers selected from the group consisting of hydrophilic polymers, hydrophobic polymers, or combinations thereof.

Suitable examples of hydrophilic rate controlling polymers include, but are not limited to cellulose derivatives such as hydroxypropylcellulose, hydroxypropylmethylcellulose, hydroxyethylcellulose, hydroxymethylcellulose, carboxymethylcellulose, methylcellulose, sodium carboxy methylcellulose or combinations thereof; polyvinylpyrrolidone, polyvinyl acetate, copolymer of vinylpyrrolidone and vinyl acetate, polysaccharides, polyalkylene glycols, starch, gums and derivatives; or mixtures thereof.

Suitable examples of hydrophobic rate controlling polymers include, but are not limited to ethyl cellulose, cellulose acetate, cellulose acetate butyrate, hydroxypropyl methylcellulose phthalate, poly (alkyl) methacrylate, and copolymers of acrylic or methacrylic acid esters, waxes, shellac and hydrogenated vegetable oils.

The composition of the present invention may be in the form of matrix-type dosage form, a reservoir type dosage form, multiple unit dosage form or combinations of these.

Matrix-type dosage forms are those in which the drug is distributed uniformly in the one or more rate controlling polymers and reservoir type dosage forms utilize polymeric coating over the core of the metaxalone. A combination of the reservoir and matrix type includes extended or sustained release coatings on extended release matrices.

Embodiments of matrix type dosage forms may include one or more following features. For example, matrix may be formulated into tablets of a single monolithic matrix, hilayered matrix or a multi-layered matrix.

Since metaxalone is indicated for acute painful musculoskeletal conditions that require quick relief it would be desirable to have a dosage form that maintains a balance between the amount of the drug released immediately and amount of drug released over an extended period.

The pharmaceutical composition comprising metaxalone of the present invention may be a bilayered matrix or a multi-layered matrix that will release metaxalone in a tailored fashion to provide both immediate and controlled release profile. The matrix may further include one or more pharmaceutically acceptable excipients.

The bilayered or multilayered tablets may optionally include a coating with one or more layers comprising film forming agents and/or pharmaceutically acceptable excipients.

In another embodiment, the metaxalone composition may be formulated as a multiple unit dosage form having plurality of discrete or aggregated particles, pellets, mini tablets, beads or granules.

The units containing metaxalone and one or more pharmaceutically acceptable polymer may include other pharmaceutically acceptable excipients. The units can be filled into a capsule or compressed into a tablet dosage form. The units prepared by the present invention may be coated with one or more layers comprising film forming agents and/or pharmaceutically acceptable excipients.

Metaxalone used in the present invention may be in one or more forms comprising metaxalone, micronized or nanonized metaxalone, metaxalone adsorbate and metaxalone-cyclodextrin admixture.

Due to poor bioavailability of metaxalone, it can be used in a micronized or nanonized form. The particle size of micronized or nanonized metaxalone may be in the range of 1 nm to 50 μm. Size reduction for micronization or nanonization may be carried out by any of the conventionally known methods using air jet mill, dyno mill, ball mill, colloid mill, grinding mill, roller mill, impact mill, etc.

Metaxalone adsorbate may be prepared by dissolving metaxalone and one or more of pharmaceutically acceptable surface modifiers in organic solvent and removing the solvent to co-precipitate the metaxalone adsorbate.

The pharmaceutically acceptable surface modifiers may be one or more of cellulose derivatives, starch, gums, sugars, saccharides, alcohols, alginates, surfactants, acrylic acid derivatives and carbohydrate based polymers.

Suitable examples of cellulosic polymers include, but are not limited to, ethylcellulose, hydmxypropyl methylcellulose, hydroxypropyl cellulose, methylcellulose, carboxymethylcellulose, cross-linked carboxymethylcellulose, hydroxymethylcellulose and hydroxyethylcellulose.

Suitable examples of acrylic acid derivatives include, but are not limited to, polymethacrylates such as ethyl acrylate/methyl methacrylate copolymer (Eudragit™ NE-30-D) and ammonio methacrylate copolymer types A and B (Eudragit™RI30D and RS30D).

Suitable surfactants can be anionic, cationic, zwitterionic and nonionic surfactants. Particularly, the compositions include at least one anionic surfactant. Suitable anionic surfactants include but are not limited to alkyl sulfonates, alkyl phosphates, alkyl phosphonates, potassium laurate, sodium lauryl sulfate, sodium dodecylsulfate, alkyl polyoxyethylene sulfates, dioctyl sodium sulfosuccinate, phosphatidyl glycerol, phosphatidylinositol, diphosphatidylglycerol, phosphatidyl inosine, phosphatidylserine, phosphatidic acid and their salts, cholic acid and other bile acids (e.g., cholic acid, deoxycholic acid, glycocholic acid, taurocholic acid, glycodeoxycholic acid) and salts thereof (e.g., sodium deoxycholate, etc.).

Suitable examples of organic solvents include, but are not limited to ketones, such as acetone; alcohols, such as methanol, ethanol, isopropyl alcohol; and chlorinated hydrocarbons, such as methylene chloride.

Solvents may be removed by techniques known in the art, for example, one or more of distillation, distillation under vacuum, evaporation, and spray drying.

Metaxalone-cyclodextrin admixture may be prepared by blending metaxalone with cyclodextrin. The admixture may also include one or more other pharmaceutically acceptable excipients.

The cyclodextrin may be a naturally occurring dextrin and also the methylated derivatives of these natural products, especially of beta-cyclodextrin.

In addition to the rate controlling polymers, the pharmaceutical composition may further include one or more pharmaceutically acceptable excipients act in one or more capacities as fillers, binders, lubricants, glidants, colorants and flavoring agents.

Suitable examples of fillers include, but are not limited to corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, sorbitol, starch, starch pregelatinized, sucrose, and mixtures thereof.

Examples of hinders include, but are not limited to methyl cellulose, hydroxypropyl cellulose, hydroxypropyl methylcellulose, polyvinylpyrrolidone, copolymer of vinyl pyrrolidone and vinyl acetate, poloxamer, gelatin, gum Arabic, ethyl cellulose, polyvinyl alcohol, pullutan, pregelatinized starch, agar, tragacanth, sodium alginate, propylene glycol, and mixtures thereof.

Examples of lubricants and glidants include, but. are not limited to colloidal anhydrous silica, stearic acid, magnesium stearate, calcium stearate, talc, hydrogenated castor oil, sucrose esters of fatty acids, microcrystalline wax, yellow beeswax, white beeswax, and mixtures thereof.

The coloring agents of the present invention may be selected from any FDA approved color for oral use.

The composition may be formulated into various pharmaceutical preparations for oral administration, e.g., in the form of a tablets, capsules, aggregated particles, pellets, mini tablets, beads or granules. The composition may be prepared in accordance with any of the conventional procedures known in the field of art, for example, simple granulation followed by sieving; extrusion and marumerization or spheronization; rotogranulation; pelletization; micropelletization, compression, coating etc. These steps may be carried out in the conventional manner.

The pharmaceutical composition comprising metaxalone prepared by the present invention may be coated with one or more layers comprising film forming agents and/or pharmaceutically acceptable excipients.

The coating layers may be applied as solution/dispersion of coating ingredients using any conventional technique known in the prior art such as spray coating in a conventional coating pan or fluidized bed processor or dip coating.

Example of solvents used for preparing a solution/dispersion of the coating ingredients include, but are not limited to, methylene chloride, isopropyl alcohol, acetone, methanol, ethanol, water and mixtures thereof.

Example of film forming agents include, but are not limited to ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate; Waxes such as polyethylene glycol; methacrylic acid polymers such as Eudragit® RL and RS; or mixture thereof. Alternatively, commercially available coating compositions comprising film-forming polymers marketed under various trade names, such as Opadry® may also be used for coating.

The pharmaceutical composition comprising metaxalone and one or more pharmaceutically acceptable polymer of the present invention have an improved pharmacokinetic profile as compared to conventional currently marketed metaxalone tablets, e.g. SKELAXIN®. For example, the improved pharmacokinetic profile of the composition of the invention may produce the same pharmacokinetic profile as a conventional metaxalone tablets (i.e. SKELAXIN®), but at a lower dose. The improved pharmacokinetic profile results in less frequent dosing as compared to a conventional metaxalone tablets, such as once a day or twice day dosing. An improved or more preferred pharmacokinetic profile to the invention may also exhibit improved Tₘₐₓ, Cₘₐₓ and/or AUC profiles.

The composition of the invention may exhibit, in comparative pharmacokinetic testing with a conventional formulation of metaxalone, such as SKELAXIN®, may exhibit a Cₘₐₓ and AUC which is greater than up to 20%, of the Cₘₐₓ and AUC exhibited by the conventional tablets of metaxalone.

The pharmaceutical composition comprising metaxalone and one or more pharmaceutically acceptable polymers of the present invention may be used for treating muscle spasm associated with painful musculoskeletal conditions in humans, that will have lower strength of drug and will provide a therapeutically effective blood concentration level of metaxalone for a sustained period of time of up to at least twelve hours.

The method may further include administering in combination with other medicines, for example, other non-steroidal anti-inflammatory agents (NSAIDs), any analgesics or other pharmaceutically active agents, etc.

The present invention is illustrated below by reference to the following example. However, it will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

### EXAMPLE

### Preparation of Metaxalone Tablets

| Ingredients | Percent w/w |
|---|---|
| Metaxalone (particle size: 19-22 μm) | 80.8 |
| | (650 mg/tablet) |
| Lactose monohydrate | 2.4 |
| Polyvinyl pyrrolidone | 6.7 |
| Copolymer of vinyl pyrrolidone and | 2.0 |
| vinyl acetate | |
| Hydroxypropyl methylcellulose | 7.1 |
| Water | q.s. |
| Stearic acid | 1.0 |

### Process:

1. Metaxalone, lactose monohydrate and hydroxypropyl methylcellulose were mixed well and granulated with solution of polyvinyl pyrrolidone and copolymer of vinyl pyrrolidone and vinyl acetate.
2. The granules were dried and lubricated with stearic acid and compressed into tablets using appropriate tooling.
   In vivo Bioequivalence Study

In vivo performance of pharmaceutical composition of metaxalone tablets prepared as per the composition of example was evaluated with respect to the SKELAXIN® 800 mg tablets in healthy male volunteers under fed condition. Two tablets (650 mg each) of Example were given as a single dose, and two tablets of SKELAXIN® 800 mg tablets were given at 6 hr interval each.

Pharmacokinetic parameters AUC₀₋₁ (Area under the plasma concentration vs. time curve from 0 hours to the time of last sample collected) and AUC_{0-inf} (Area under the plasma concentration vs time curve from 0 hours to infinity) were calculated from the data obtained. Statistical analysis was carried out at 90% confidence interval using “SAS” software package. The results of the study are given in Table 1.

TABLE 1
Pharmacokinetic data for tablets of Example (650 mg/tablet) vs.
SKELAXIN ® (800 mg/tablet)

| Pharmacokinetic | Pharmacokinetic parameters | |
|---|---|---|
| parameters (log | of Example (650 mg tablets) | |
| transformed) | Ratio (% reference) | 90% Confidence Interval |
| AUC₀₋ₜ | 101.06 | 87.89-116.21 |
| AUC_{0-inf} | 100.95 | 88.23-115.50 |

The AUC parameters of tablets of Example (650 mg/tablet) exhibited comparable values to the conventional metaxalone tablets (800 mg/tablet).

While there has been shown and described what are the preferred embodiments of the invention, one skilled in the pharmaceutical formulation art will appreciate that various modifications in the formulations and process can be made without departing from the scope of the invention as it is defined by the appended claims.

1. A pharmaceutical composition for oral administration of a low-dose of metaxalone comprising
metaxalone and
one or more pharmaceutically acceptable polymers,
wherein the daily dose of metaxalone is reduced by at least 10% of the conventional daily dose.

2. The composition according to claim 1, wherein the dose of metaxalone is reduced by at least 15% of the conventional daily dose.

3. The composition according to claim 1, wherein the daily dose of metaxalone is from about 2000 mg to about 2900 mg.

4. The composition according to claim 3, wherein the daily dose of metaxalone is from about 2400 mg to about 2600 mg

5. The composition according to claim 1 or 2, wherein the low-dose metaxalone pharmaceutical composition is administered twice a day and exhibits pharmacokinetic parameters comparable to the conventional dosage form (SKELAXIN® 800 mg) administered four times a day.

6. The composition according to claim 1, wherein the polymer is rate controlling polymer selected from hydrophilic polymers, hydrophobic polymers or combinations thereof.

7. The composition according to claims 1 and 2, wherein the composition is one of matrix-type dosage form, a reservoir type dosage form, multiple unit dosage form or combinations of these.

8. The composition according to claim 7, wherein the matrix type dosage form comprises single monolithic matrix, bilayered matrix and multi-layered matrix.

9. The composition according to any of the preceding claims wherein the metaxalone is in one or more forms comprising metaxalone, micronized or nanonized metaxalone, metaxalone adsorbate and metaxalone-cyclodextrin admixture.

10. The composition according to claim 6, wherein the hydrophilic rate controlling polymers comprise one or more of hydroxypropylcellulose, hydroxypropylmethylcellulose, hydroxyethylcellulose, hydroxymethylcellulose, carboxymethylcellulose, methylcellulose, sodium carboxy methylcellulose, polyvinylpyrrolidone, polyalkylene glycols, starch, gums and derivatives or mixtures thereof.

11. The composition according to claim 6, wherein the hydrophobic rate controlling polymers comprise one or more of ethyl cellulose, cellulose acetate, cellulose acetate butyrate, hydroxypropyl methylcellulose phthalate, poly (alkyl) methacrylate, and copolymers of acrylic or methacrylic acid esters, waxes, shellac, hydrogenated vegetable oils and mixtures thereof.

12. The composition according to any of the preceding claims wherein the composition further comprises pharmaceutically acceptable excipients of one or more of fillers, binders, lubricants, glidants, colorants and flavoring agents.

13. (canceled)

14. The composition according to claim 12, wherein the composition comprise one or more layers of film forming agents.

15. A method of treating muscle spasm associated with painful musculoskeletal conditions in humans, by administering a pharmaceutical composition comprising low-dose of metaxalone and one or more pharmaceutically acceptable polymer according to claim 1 to 2.

16. The method of treatment according to claim 14, wherein the pharmaceutical composition of metaxalone further comprises other non-steroidal anti-inflammatory agents (NSAIDs), analgesics and other pharmaceutical agents.
